(19) United States
(12) Patent Application Publication
(10) Pub. No.: US 2024/0165179 A1
(43) Pub. Date: May 23, 2024

(54) Cordyceps Militaris Designated as Strain M2-105-04, Compositions, Methods of Making, Methods of Using and Methods for Treating Mammal

(71) Applicant: M2 Ingredients, Inc., Vista, CA (US)

(72) Inventors: Samuel Andrasko, Lakeside, CA (US); Julie Daoust, Vista, CA (US); Steven Lee Farrar, Vista, CA (US)

(21) Appl. No.: 18/516,756

(22) Filed: Nov. 21, 2023

Related U.S. Application Data
(60) Provisional application No. 63/427,726, filed on Nov. 23, 2022.

(51) Int. Cl. A61K 36/068

(52) U.S. Cl. CPC A61K 36/068; A61K 2236/11; A61K 2236/15; A61K 2236/17

(57) ABSTRACT

Exemplary embodiments include a whole mycelium of Cordyceps militaris designated as strain M2-105-04 as deposited with the ATCC Patent Depository under the Budapest Treaty (10801 University Blvd, Manassas, VA 20110), on Jul. 28, 2023, with the unofficial ATCC Patent Deposit Designation No. PTA-127612 and the official deposit date of Jul. 28, 2023 and the official patent deposit number of PTA-127612. The whole mycelium is characterized by a phenotype having an abundance of fruiting bodies on top of a substrate and/or along a parameter of a bioreactor bag. It is also characterized by a phenotype having fruiting initiation at or near 18 days after inoculation, a phenotype having fruiting bodies maturing at or near 55 days after inoculation, and/or a phenotype having a 10-to-15-day colonization time on hulled oats.

### CROSS-REFERENCE TO RELATED APPLICATIONS

This Non-Provisional Patent application claims the priority benefit of U.S. Provisional Patent Application Ser. No. 63/427,726 filed on Nov. 23, 2022, and titled “Cordyceps Militaris Designated as Strain M2-105-04,” the entirety of which is hereby incorporated by reference and including all appendices and patent deposit material(s).

### FIELD OF THE TECHNOLOGY

Embodiments of the disclosure relate to an improved strain of Cordyceps militaris.

### BRIEF SUMMARY OF EXEMPLARY EMBODIMENTS

Cordyceps militaris is difficult to cultivate on a large scale. Additionally, the fruiting bodies of Cordyceps militaris are relatively small, which makes them time-consuming to harvest. The amount of active compounds in Cordyceps militaris can vary depending on the growing conditions, the extraction process, and the storage conditions. Consistency is key. The exemplary embodiments herein solve these problems.

Exemplary embodiments include a whole mycelium of Cordyceps militaris designated as strain M2-105-04 as deposited with the ATCC Patent Depository under the Budapest Treaty (10801 University Blvd, Manassas, VA 20110), on Jul. 28, 2023, with the unofficial ATCC Patent Deposit Designation No. PTA-127612 and the official deposit date of Jul. 28, 2023 and the official patent deposit number of PTA-127612. The whole mycelium is characterized by a phenotype having an abundance of fruiting bodies on top of a substrate and/or along a parameter of a bioreactor bag. It is also characterized by a phenotype having fruiting initiation at or near 18 days after inoculation, a phenotype having fruiting bodies maturing at or near 55 days after inoculation, and/or a phenotype having a 10-to-15-day colonization time on hulled oats.

According to various exemplary embodiments, the whole mycelium is characterized by a phenotype optimized to grow on oats. The whole mycelium may also be characterized by a phenotype having a beta-glucan content of approximately 20% to 25%. In various exemplary embodiments, the whole mycelium is characterized by a phenotype having a beta-glucan content of approximately 26% to 31%. In other exemplary embodiments, the whole mycelium is characterized by a phenotype having a beta-glucan content of approximately 32% to 37%, a phenotype having a beta-glucan content of approximately 38% to 43%, and/or a phenotype having a beta-glucan content of approximately 44% to 49%.

The whole mycelium, according to some exemplary embodiments, may be in a powder form, a capsule form and/or in a drink mix form for cold and hot beverages applications.

Exemplary methods for propagating a Cordyceps militaris mycelium includes the steps of:
culturing a mother culture;
culturing a production culture;
creating a liquid master inoculum;
diluting the liquid mater inoculum;
creating a biomass product;
slicing the biomass product;
dehydrating the biomass product; and
milling the dehydrated biomass product.

### BRIEF DESCRIPTION OF THE DRAWINGS

The patent or application file contains at least one photograph executed in color. Copies of this patent or patent application publication with color photograph(s) will be provided by the Office upon request and payment of the necessary fee.

While this technology is susceptible of embodiment in many different forms, there is illustrated in the drawings, and will herein be described in detail, several specific embodiments with the understanding that the present disclosure is to be considered as an exemplification of the principles of the technology and is not intended to limit the technology to the embodiments illustrated.

It will be understood that like or analogous elements and/or components, referred to herein, may be identified throughout the drawings with like reference characters. It is further understood that several of the figures are merely schematic representations of the present technology. As such, some of the components may be distorted from their actual scale for pictorial clarity.

FIG. 1 shows an exemplary method for propagating a Cordyceps militaris mycelium.

FIG. 2 is a color photograph showing a fruitbody perithecia close up of Cordyceps militaris.

FIG. 3 is a color photograph showing a fruitbody perithecia of Cordyceps militaris.

FIG. 4 is a color photograph showing a full bioreactor bag of Cordyceps militaris.

FIG. 5 is a color photograph showing a full interior of a bioreactor bag of Cordyceps militaris.

FIG. 6 is a color photograph showing a natural phenotype of FIG. 7 is a color photograph showing a production bioreactor bag of Cordyceps militaris.

FIG. 8 is a color photograph showing a surrounding fruitbody interior of a bioreactor bag of Cordyceps militaris.

### DETAILED DESCRIPTION OF EXEMPLARY EMBODIMENTS

Exemplary embodiments include a whole mycelium of Cordyceps militaris designated as strain M2-105-04 as deposited with the ATCC Patent Depository under the Budapest Treaty (10801 University Blvd, Manassas, VA 20110), on Jul. 28, 2023, with the unofficial ATCC Patent Deposit Designation No. PTA-127612.

The whole mycelium is characterized by a phenotype having an abundance of fruiting bodies on top of a substrate and along a parameter of a bioreactor bag. It is also characterized by a phenotype having fruiting initiation at or near 18 days after inoculation, and a phenotype having fruiting bodies maturing at or near 55 days after inoculation. The phenotype may be optimized to grow on oats and may have a 10-to-15-day colonization time on hulled oats.

According to various exemplary embodiments, an organic grain substrate is a sterile, nutrient-rich medium used to grow Cordyceps militaris. It may be composed of a combination of whole grains, such as rice, rye, oats, or wheat, supplemented with other organic ingredients like wheat bran, brewer's yeast, and calcium carbonate. These substrates provide the necessary nutrients and support for the mycelium, the vegetative part of the Cordyceps militaris, to grow and develop into fruiting bodies.

In various exemplary embodiments, organic grain substrates are preferred over non-organic substrates due to several advantages, including reduced risk of contamination. Organic grains are less likely to contain harmful chemicals or pathogens that could contaminate the Cordyceps militaris. They also have enhanced nutritional value. Organic grains are generally higher in nutrients and minerals, which can contribute to the nutritional value of the Cordyceps militaris grown on them.

Additionally, in certain exemplary embodiments, the substrate should be sterilized to eliminate any potential contaminants before inoculating it with Cordyceps militaris spawn. The substrate should have a balanced moisture content to support optimal mycelium growth and comprise a balanced blend of nutrients to provide the Cordyceps militaris with essential elements for healthy development.

In exemplary embodiments, the whole mycelium may be characterized by a phenotype having a cordycepin content of approximately 0.4 milligrams per gram to 1.1 milligrams per gram as represented relative to the total mycelium and fruiting bodies. The whole mycelium, in other exemplary embodiments, may be characterized by a phenotype having a cordycepin content of approximately 0.4 milligrams per gram to 0.75 milligrams per gram as represented relative to the total mycelium and fruiting bodies, or the whole mycelium may be characterized by a phenotype having a cordycepin content of approximately 0.75 milligrams per gram to 1.1 milligrams per gram as represented relative to the total mycelium and fruiting bodies in other exemplary embodiments.

Beta-glucan, according to various exemplary embodiments, is a type of soluble fiber found in the cell walls of Cordyceps militaris. It is a natural compound with a wide range of potential health benefits. Beta-glucan has been shown to have a number of potential health benefits, including immune system support. Beta-glucan is a strong immunostimulant, meaning it can help to boost the immune system's ability to fight off infection. It does this by activating certain immune cells, such as macrophages and natural killer cells. Beta-glucan can help to lower cholesterol levels by binding to cholesterol in the digestive tract and preventing it from being absorbed into the bloodstream. This can help to reduce the risk of heart disease. Beta-glucan can help to regulate blood sugar levels by slowing down the absorption of sugar into the bloodstream. This can be beneficial for people with diabetes or prediabetes. Beta-glucan is a prebiotic, which means it is a type of fiber that can promote the growth of beneficial bacteria in the gut. This can help to improve digestive health and reduce the risk of digestive problems.

In further exemplary embodiments, the phenotype may have a beta-glucan content of approximately 20% to 25%, approximately 26% to 31%, approximately 32% to 37%, approximately 38% to 43%, and/or approximately 44% to 49%, as represented as a percentage of the total mycelium and fruiting bodies. The mycelium and fruiting bodies may be substantially dehydrated and in a powder form, a capsule form and/or in a drink mix powder for cold beverages application or a drink mix form for a hot beverage application.

An exemplary method for propagating a Cordyceps militaris mycelium may include the steps of culturing a mother culture, culturing a production culture, creating a liquid master inoculum, diluting the liquid master inoculum, creating a biomass product, slicing the biomass product, dehydrating the biomass product, and/or milling the dehydrated biomass product.

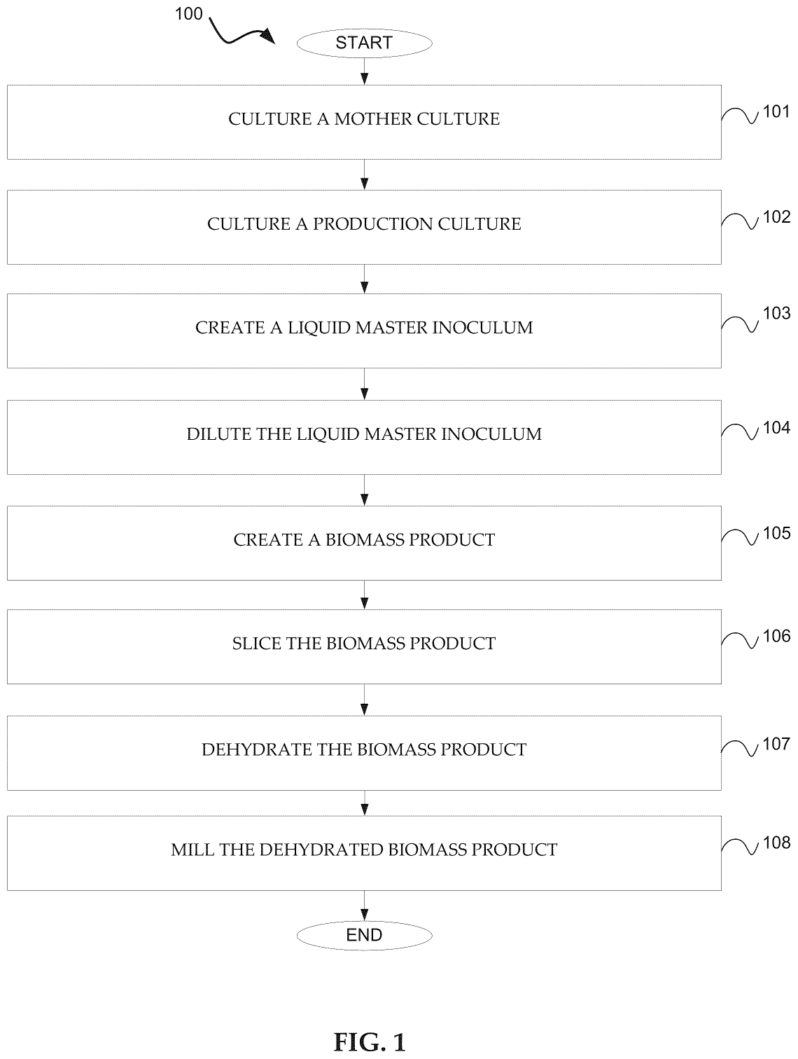

FIG. 1 shows an exemplary method 100 for propagating a Cordyceps militaris mycelium.

At step 101, a mother culture or preserved stock culture is cultured.

According to exemplary embodiments, fresh cultures of the Cordyceps militaris mycelium of the M2-105-04 strain is cultured on nutrified agar and grown for a period of 7-10 days until a culture diameter of 30-40 mm is obtained. 5 mm×5 mm square sections of the healthiest part of the culture is cut from the leading edge of growth and placed into a 1.5 ml-2 ml cryogenic storage vial of 10-20% glycerol solution and cryogenically stored under LN2 (liquid nitrogen). At the same time, 2 mm square sections of the same culture material is transferred to 10-20 culture tubes=>25 mm×150 mm in size and filled with a nutrient agar comprising a carbon/nitrogen/vitamin/mineral mix and subsequently stored under refrigeration between 34-40 degrees Fahrenheit. Culture tubes are sub-cultured every 6-12 months to new culture tubes to verify that the cultures are still active and healthy and to preserve the phenotypic qualities of the original cultures. Cryogenic cultures are recovered and sub-cultured into new culture tubes for storage and not used if any unwanted phenotypical variation is observed. Only growth representative of the mother culture is used for inoculum.

At step 102, a production culture is cultured.

To start the production process, according to various exemplary embodiments, mother cultures are pulled from refrigeration and sub-cultured onto 4 nutrient agar plates. Cultures are allowed to grow for 9-16 days until a growth diameter of 30-40 mm is obtained. The healthiest and most representative tissue of the mother culture is sub-cultured at step 103.

At step 103, a liquid master inoculum is created.

Nutrified liquid media is filled into 2 liter flasks and sterilized. They are then placed into a HEPA filtrated laminar flow hood to cool. Once cooled, a laboratory technician subcultures a 10 mm×10 mm section of the healthiest tissue from step 102 to each individual flask. The flasks are then stirred and incubated for 7-14 days under darkness and subsequently placed under refrigeration to prevent the culture from growing/fruiting further until use.

At step 104, the liquid master inoculum is diluted.

One day before biomass inoculation, a 10-liter carboy fitted with a port cap and a luer-lock dispensing system is sterilized with 8 liters of H₂0 and sterilized inside of an autoclave. The carboy is allowed to cool in front of a HEPA filtrated laminar flow hood. Once cooled, a laboratory technician dilutes the master liquid spawn into the 8 liters of H₂0 creating a 10-liter dilution solution of the inoculum. The inoculum is now ready for biomass inoculation.

At step 105, a biomass product is created.

According to exemplary embodiments, 600 to 800 units of 8.5 pounds each of hydrated hulled oats, hydrated to a 50-60% moisture content in static and/or dynamic water tanks @190-205 Fahrenheit for 30 min to 60 min is filled into an appropriate clear autoclavable bioreactor bag comprised of a polypropylene or high-density polyethylene or a polypropylene/high density polyethylene blend fitted with a microporous filter patch or strip material for gas exchange. The oats are subjected to sterilization in an autoclave for an appropriate time and temperature to fully render any present microorganisms inactive. The bags are cooled in a HEPA filtrated refrigerated cooldown room until the grain is =<90 Fahrenheit. Once cooled, the bags are delivered to a HEPA filtrated laminar flow hood and a laboratory technician transfers the dilution solution to the biomass bags at approximately 5 to 12.5 milliliters per bag. The bags are then incubated for approximately 35 to 55 days. The bags may comprise the fruiting bodies, mycelium and digested oats.

At step 106, the biomass product is sliced.

Upon harvesting the biomass, in various exemplary embodiments, it is sliced though a dicer into smaller pieces and placed upon dehydration trays for dehydration.

At step 107, the biomass product is dehydrated.

In various exemplary embodiments, the trays of biomass product are loaded into a static dehydrator and dried for approximately 24 hours and/or into a dynamic continuous dehydrator for approximately 30-60 minutes to a moisture content of <5%. This process is carefully watched to prevent altering the taste of the final product.

At step 108, the dehydrated biomass product is milled.

The dried biomass is put though a mill and screened to a designated particle size and bulk packed for customers.

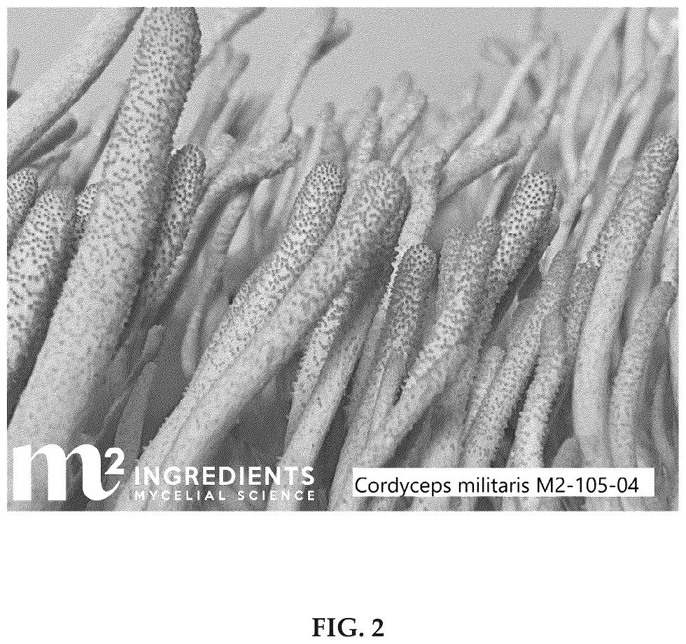

FIG. 2 is a color photograph showing a fruitbody perithecia close up of Cordyceps militaris.

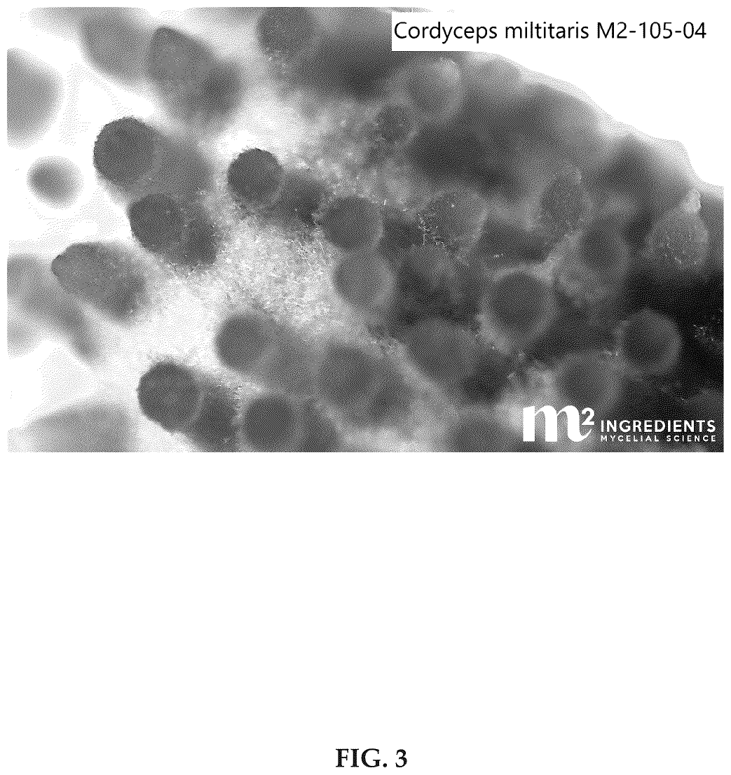

FIG. 3 is a color photograph showing a fruitbody perithecia of Cordyceps militaris.

In mycology, a perithecium (plural perithecia) is a flask-shaped fruiting body, also known as an ascocarp, produced by some ascomycete fungi. It is a type of sexual reproductive structure that houses the asci, which are the sac-like structures that produce ascospores, the sexual spores of ascomycete fungi.

Perithecia are typically small, ranging in size from a few micrometers to a few millimeters in diameter. They can be single-celled or multi-celled, and they may have a variety of shapes, including spherical, flask-shaped, or club-shaped.

The perithecium is formed from hyphae, which are the thread-like filaments that make up the fungal body. The hyphae intertwine to form a protective outer layer, called the peridium. Inside the peridium, a layer of cells, called the ascogenous layer, develops. The ascogenous layer is responsible for producing the asci, which contain the ascospores.

When the asci are mature, they release the ascospores through a small opening in the peridium. The ascospores can then be carried by air or water to new locations, where they can germinate and grow into new fungal colonies.

The term perithecium is derived from the Greek words peri (around) and theke (receptacle), reflecting the flask-shaped structure of the reproductive body.

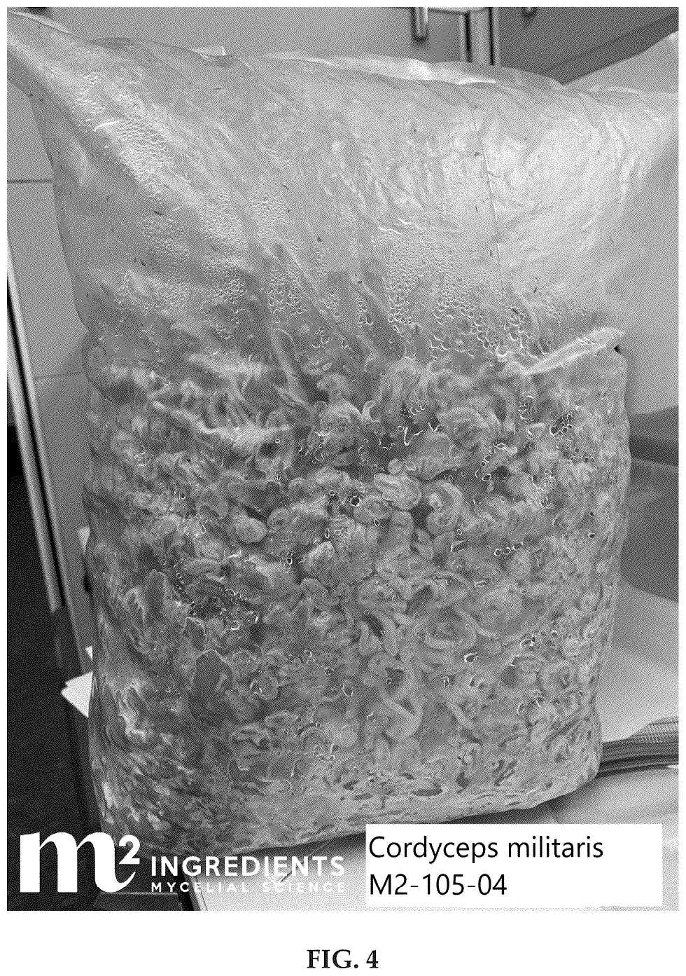

FIG. 4 is a color photograph showing a full bioreactor bag of Cordyceps militaris.

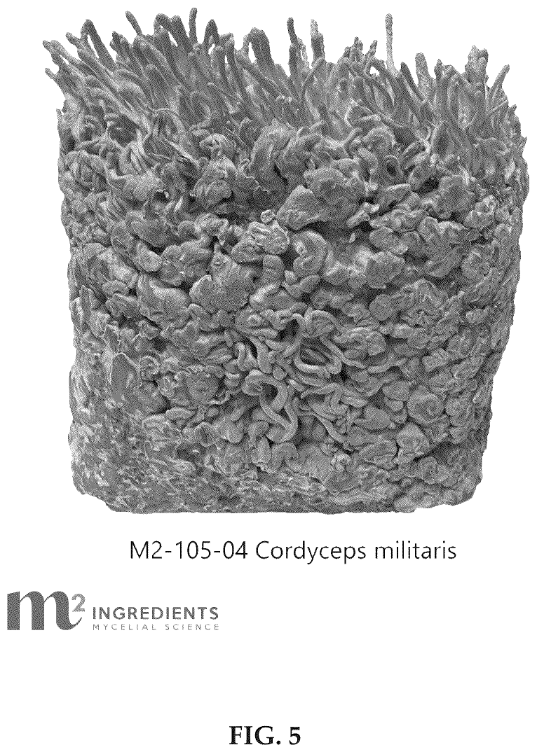

FIG. 5 is a color photograph showing a full interior of a bioreactor bag of Cordyceps militaris.

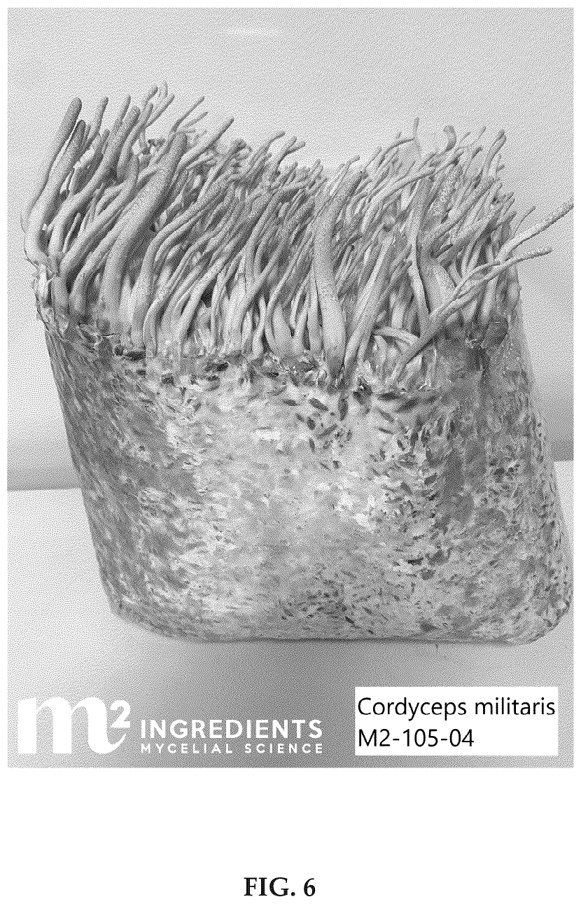

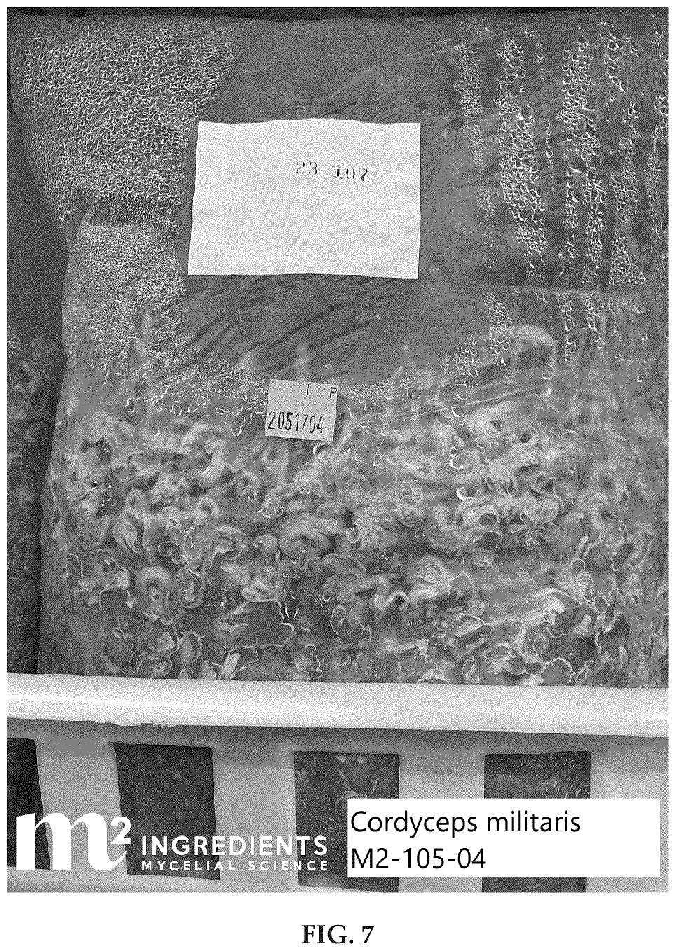

FIG. 6 is a color photograph showing a natural phenotype of Cordyceps militaris.

FIG. 7 is a color photograph showing a production bioreactor bag of Cordyceps militaris.

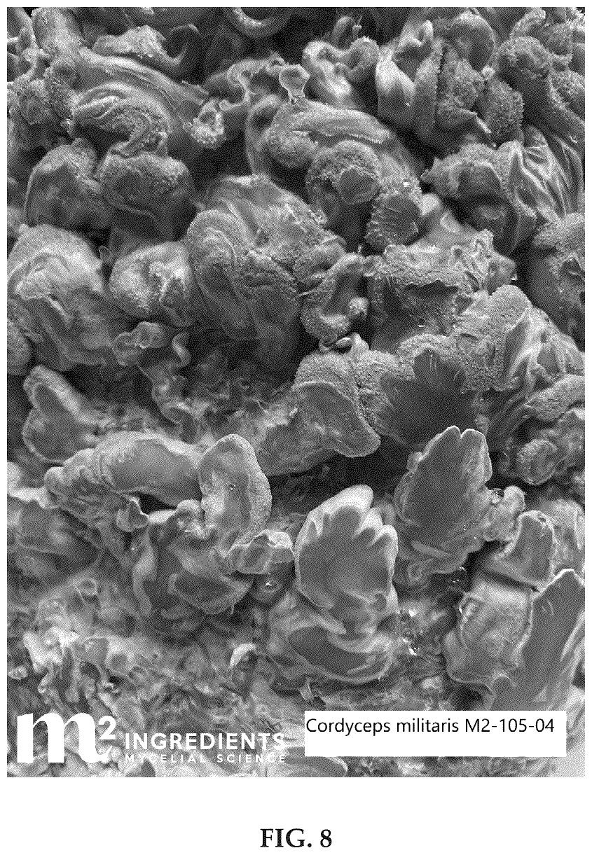

FIG. 8 is a color photograph showing a surrounding fruitbody interior of a bioreactor bag of Cordyceps militaris.

According to various exemplary embodiments, a bioreactor bag is a sterile, flexible bag that is used to culture Cordyceps militaris. Bioreactor bags provide a controlled environment for the growth of Cordyceps militaris, and they can be used to produce large quantities of Cordyceps militaris in a relatively small space.

Bioreactor bags are typically made of polypropylene or high density polythylene film. They have a number of features that make them suitable for culturing microorganisms, including a sterile interior to prevent contamination, a gas exchange port to allow for the exchange of oxygen and carbon dioxide.

Additional information.

Master culture storage parameters may include media additives such as yeast, rice protein, and cauliflower powder. Antibiotics including gentamicin and chloramphenicol may be used to ensure the culture is clean and free of contamination as a quality control measure. Subzero storage is acceptable, and storage may take place at 36 to 40 Fahrenheit (F).

Culturing parameters may include an incubation temperature of approximately 68 to 72 Fahrenheit in no or little light. Culturing times may include approximately 7 to 12 days with a 100-millimeter (MM) plate, approximately 5 days with a 25 MM×150 MM tube, and approximately 7 days with 500 milliliters (ml) of liquid. Culturing parameters may include media additives such as yeast, rice protein, and cauliflower powder.

Master spawn parameters may include an incubation temperature of approximately 68 to 72 Fahrenheit in no or little light. Culturing time may be approximately 7 to 14 days with liquid culture. Culturing parameters may include media additives such as yeast, rice protein, and cauliflower powder. Storage may take place at 36 to 40 Fahrenheit (F).

Production spawn parameters may include media of oats and sorghum with a large bioreactor (8 pounds) for approximately 10 days in little or no light. The culture may be shaken on approximately day 6. Storage may take place at 36 to 40 Fahrenheit (F).

Biomass product parameters may include oats as media, incubation temperature of approximately 68 to 72 F, with approximately 54 days in a large bioreactor (8 pounds). Light is provided at approximately 12 hours on and 12 hours off. The 12 hours of on light intensity may be at an intensity of 300 to 500 lux, wavelength blue at approximately 450 nm plus red at 650 nm.

In various exemplary embodiments, a peristaltic pump may be used for dispensing liquid inoculum into bioreactor bags filled with grain media. A bioreactor bag is an autoclavable HDPE, PP or HDPE/PP blend bag equipped with membrane air filter to allow mushroom culture to breathe during the incubation period. Inoculation is a key step in the production process and inattention to procedures and/or aseptic technique can result in microbial contamination and the loss of the entire batch.

Hydration of organic grain (substrate) may take place in static hydration tanks and/or dynamic hydration tanks prior to the semi-automated bag filling and sterilization operations. It is typically a first step in the processing of the substrate used for the solid-state fermentation of mycelial biomass for Cordyceps militaris.

### Example One

As reflected in Appendix 1, a study was conducted in accordance to the OECD Guideline for the testing chemical No. 425 to determine the LD —50 of the Cordyceps Mushroom Powder (Strain M2-105-04). The Cordyceps Mushroom Powder consisted of pure Cordyceps mycelium and fruiting bodies. Nothing else was added.

The test sample was suspended in the purified water. The test was conducted in 2 parts—Limit Test & the Main Test. In the limit test 5 animals were treated with 2000 mg/kg/body weight of the sample orally in female Wistar rats (8-12 weeks) by oral gavage. The body weight of animals was measured at day 0 (before the drug administration) and on days 1, 7, and 14 of the study. The animals were observed for 48 hours for mortality. No mortality was observed in the animals in 48 hours or at a later stage. In the second stage the Main Test 3 animals were taken for each dose group and treated in a downward dosing of 625, 195 and 61 mg/kg/body weight and observed for mortality. Cage side observations included variations in the skin and fur, eyes, and mucous membranes. Particular attention was directed to observations of tremor, convulsions, salivation, diarrhea, lethargy, sleep, and coma. Also, respiratory, circulatory, autonomic, and central nervous systems and somatomotor activity were examined.

In the study, no mortality was observed up to 14 days. No toxic symptoms were found in rats in all four doses. No abnormalities were observed in general clinical observations or gross necropsy. The rats were found to behave normally with no variation in locomotors, behavioral, neurological, or secretary patterns. No significant changes were observed in the body weight of rats in all four treated doses.

Considering the data obtained from the study, the Cordyceps Mushroom Powder sample is not found to be lethal up to a dose of 2000 mg/kg and had an LD-50 of above 2000 mg/kg body weight. According to the GHS (Globally Harmonized Classification System), the test sample Cordyceps Mushroom Powder was classified into Category 4.

### Example Two

As reflected in Appendix 2, a study was conducted in accordance to the OECD Guideline for the testing chemical No. 408 to evaluate the Repeated dose 90 days Sub Chronic Oral Toxicity of Cordyceps Mushroom Powder (Strain M2-105-04). The Cordyceps Mushroom Powder consisted of pure Cordyceps mycelium and fruiting bodies. Nothing else was added.

The test sample, Cordyceps Mushroom Powder (Strain M2-105-04) was administered orally using oral gavage. Dose is expressed as weight (g, mg) or as weight of the test substance per unit weight of test animal (e.g. mg/kg). In this test we have taken 100 Wistar rats (50 male & 50 female) divided into 10 groups (5 female groups and 5 male groups) each group containing 10 animals.

The body weight of animals was measured at day 0 (before the drug administration) and on every 12th day till 90 days of the study. The rate of mortality and morbidity of the animals were observed every day continuously for 90 days. Opthalmological examination, and motor coordination activity were observed on every week of the study. Cage side observations included variations in the skin and fur, eyes, and mucous membranes.

In the study, no mortality and morbidity were observed up to 90 days. No toxic symptoms were found in rats in all six doses. No abnormalities were observed in general clinical observations or gross necropsy. The rats were found to behave normally with no variation in the haematology, clinical biochemistry and histopathological studies. No significant changes were observed in the body weight of rats in all six treated doses.

Considering the data obtained from the study, the Cordyceps Mushroom Powder (Strain M2-105-04) Mushroom Powder (Strain M2-105-04 is found to be non-toxic up to a dose of 2000 mg/kg. According to the GHS (Globally Harmonized Classification System), the test sample Cordyceps Mushroom Powder (Strain M2-105-04) Mushroom Powder (Strain M2-105-04 was classified into Category 4.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the technology as defined by the appended claims. Thus, the breadth and scope of a preferred embodiment should not be limited by any of the above-described exemplary embodiments.

What is claimed is:

1. A whole mycelium of Cordyceps militaris designated as strain M2-105-04 as deposited with the ATCC Patent Depository under the Budapest Treaty (10801 University Blvd, Manassas, VA 20110), on Jul. 28, 2023, with the unofficial ATCC Patent Deposit Designation No. PTA-127612 and the official deposit date of Jul. 28, 2023 and the official patent deposit number of PTA-127612.

2. The whole mycelium of claim 1 characterized by a phenotype having an abundance of fruiting bodies on top of a substrate and along a parameter of a bioreactor bag.

3. The whole mycelium of claim 1 characterized by a phenotype having fruiting initiation at or near 18 days after inoculation.

4. The whole mycelium of claim 1 characterized by a phenotype having fruiting bodies maturing at or near 55 days after inoculation.

5. The whole mycelium of claim 1 characterized by a phenotype having a 10-to-15-day colonization time on hulled oats.

6. The whole mycelium of claim 1 characterized by a phenotype optimized to grow on oats.

7. The whole mycelium of claim 1 characterized by a phenotype having a beta-glucan content of approximately 20% to 25% as represented as a percentage of the total mycelium and fruiting bodies.

8. The whole mycelium of claim 1 characterized by a phenotype having a beta-glucan content of approximately 26% to 31% as represented as a percentage of the total mycelium and fruiting bodies.

9. The whole mycelium of claim 1 characterized by a phenotype having a beta-glucan content of approximately 32% to 37% as represented as a percentage of the total mycelium and fruiting bodies.

10. The whole mycelium of claim 1 characterized by a phenotype having a beta-glucan content of approximately 38% to 43% as represented as a percentage of the total mycelium and fruiting bodies.

11. The whole mycelium of claim 1 characterized by a phenotype having a beta-glucan content of approximately 44% to 49% as represented as a percentage of the total mycelium and fruiting bodies.

12. The whole mycelium of claim 1, the whole mycelium characterized by a phenotype having a cordycepin content of approximately 0.4 milligrams per gram to 1.1 milligrams per gram as represented relative to the total mycelium and fruiting bodies.

13. The whole mycelium of claim 1 wherein the whole mycelium is in a powder form.

14. The whole mycelium of claim 13 wherein the whole mycelium is a capsule form.

15. The whole mycelium of claim 13 wherein the whole mycelium is in a drink mix form for cold and hot beverages applications.

16. A method for propagating a Cordyceps militaris mycelium comprising the steps of:
a. culturing a mother culture;
b. culturing a production culture;
c. creating a liquid master inoculum;
d. diluting the liquid mater inoculum;
e. creating a biomass product;
f. slicing the biomass product;
g. dehydrating the biomass product; and
h. milling the dehydrated biomass product.

17. A method for treating a mammal to improve immune functioning, the method comprising administering an effective dose of Cordyceps Mushroom Powder consisting of pure Cordyceps mycelium and fruiting bodies, the effective dose being administered once every 12 days over 8 separate consecutive 12-day intervals.

18. The method of claim 17, further comprising the effective dose being 500 mg of the Cordyceps Mushroom Powder per kg of body weight of the mammal.

19. The method of claim 17, further comprising the effective dose being 1000 mg of the Cordyceps Mushroom Powder per kg of body weight of the mammal.

20. The method of claim 17, further comprising the effective dose being 2000 mg of the Cordyceps Mushroom Powder per kg of body weight of the mammal.

21. The method of claim 17, further comprising the mammal being a rat.

22. The method of claim 17, further comprising the mammal being a human.
